(19) United States
(12) Patent Application Publication
(10) Pub. No.: US 2024/0268873 A1
(43) Pub. Date: Aug. 15, 2024

(54) BONE CUTTING GUIDE SYSTEMS AND METHODS

(71) Applicant: Treace Medical Concepts, Inc., Ponte Vedra, FL (US)

(72) Inventors: Paul Dayton, Ankeny, IA (US); Joe William Ferguson, Ponte Vedra Beach, FL (US); F. Barry Bays, Collierville, TN (US); John T. Treace, Ponte Vedra Beach, FL (US); W. Bret Smith, Lexington, SC (US)

(21) Appl. No.: 18/642,470

(22) Filed: Apr. 22, 2024

Related U.S. Application Data
(63) Continuation of application No. 17/106,306, filed on Nov. 30, 2020, now Pat. No. 11,963,703.
Application No. 17/106,306 is a continuation of application No. 15/210,497, filed on Jul. 14, 2016, now Pat. No. 10,849,663.
(60) Provisional application No. 62/192,290, filed on Jul. 14, 2015.

(51) Int. Cl. A61B 17/80; A61B 17/15; A61B 17/56

(52) U.S. Cl. CPC A61B 17/8061; A61B 17/15; A61B 17/151; A61B 2017/564

(57) ABSTRACT

A bone cutting guide may include a support that contains a shaft movable relative to the support. The shaft may carry a guide member having one or more cut guides through which a clinician inserts a cutting member to cut bone positioned under the guide cut guides. In operation, a clinician may fixate the support of the bone cutting guide to a bone and translate the guide member until the one or more cut guides are positioned at a desired cut location. The clinician may then perform a cut through the cut guide.

### RELATED APPLICATION

This application is a continuation of U.S. patent application Ser. No. 17/106,306, filed Nov. 30, 2020, which is a continuation of U.S. patent application Ser. No. 15/210,497, filed Jul. 14, 2016 and issued as U.S. Pat. No. 10,849,663 on Dec. 1, 2020, which claims the benefit of U.S. Provisional Application Ser. No. 62/192,290, filed Jul. 14, 2015. The entire contents of these applications are hereby incorporated by reference.

### TECHNICAL FIELD

This disclosure relates generally to devices and methods for cutting bones.

### BACKGROUND

Bones, such as the bones of a foot, may be anatomically misaligned. In certain circumstances, surgical intervention is required to correctly align the bones to reduce patient discomfort and improve patient quality of life.

### SUMMARY

In general, this disclosure is directed to bone cutting guide systems and techniques for cutting bones. In some examples, a bone cutting guide includes a support that houses a shaft that can translate relative to the support. The shaft may carry a main guide member that defines one or more cutting guide surfaces. For example, the main guide member may define opposed guide surfaces configured to receive a cutting member. In use, the cutting member may be inserted between the opposed guide surfaces and bounded within a range of movement by the guide surfaces, causing the cutting member to be directed at a cutting location under the guide surfaces. Additionally or alternatively, the main guide member may define a single cutting surface/plane. The cutting surface/plane may be a surface against which a clinician can position a cutting member and then guide the cutting member along the cutting surface/plane to perform a cutting operation.

In some configurations, the bone cutting guide includes fixation members, such as fixation pins or apertures, that allow the main body to be fixated on or adjacent a bone to be cut. For example, in use, a clinician may fixate the main body to a bone (e.g., a first metatarsal). Thereafter, the clinician may translate the main guide member having at least one cutting guide surface (e.g., opposed cutting guide surfaces) relative to the fixed main body. The clinician can translate the main guide member by sliding or rotating the shaft housed within the main body, e.g., causing the distal end of the shaft and main guide member carried thereon away from or towards the main body. Once suitably positioned, the clinician may or may not lock the location of the shaft and perform one or more cuts through the guide surfaces of the main guide member.

To perform a surgical procedure, a clinician may attach the support of the bone cutting guide to a bone. For example, the clinician may insert fixation members, such as fixation pins or screws, through apertures in the support to fixate the support to the bone (e.g., first metatarsal). Thereafter, the clinician may translate the main guide member having at least one cutting guide surface (e.g., opposed cutting guide surfaces) relative to the support. The clinician can translate the main guide member by moving a shaft housed within the inner cavity of the support, e.g., causing the distal end of the shaft and main guide member carried thereon to move away from or towards the support. Once suitably positioned, the clinician may or may not lock the location of the shaft and perform one or more cuts through the guide surfaces of the main guide member.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 is a perspective view of a bone cutting guide in accordance with an embodiment of the invention.

FIG. 2 is a perspective view of the bone cutting guide of FIG. 1, with a spacer detached.

FIG. 3 is a top view of the bone cutting guide of FIG. 1.

FIG. 4 is a top view of the bone cutting guide of FIG. 1, with the spacer removed.

FIG. 5 is a side perspective view of a bone cutting guide on a foot in accordance with a medical procedure of an exemplary embodiment of the invention.

FIG. 6 is a side perspective view of a bone cutting guide on a foot held by fixation pins and positioned for a first bone cut in accordance with an embodiment of the invention.

FIG. 7 is a perspective view of a bone cutting guide and a bone positioning guide on a foot in accordance with an embodiment of the invention.

FIG. 8 is a perspective view of a bone cutting guide and a bone positioning guide on a foot depicting a bone adjustment in accordance with an embodiment of the invention.

FIG. 9 is a perspective view of a bone cutting guide and a bone positioning guide on a foot depicting a bone adjustment in accordance with an embodiment of the invention.

FIG. 10 is a perspective view of a bone cutting guide and a bone positioning guide on a foot positioned for a second bone cut in accordance with an embodiment of the invention.

FIG. 11 is a perspective view of a bone positioning guide on a foot in accordance with an embodiment of the invention.

FIG. 12 is a side perspective view of a bone positioning guide on a foot depicting an olive pin providing compression between first and second bones in accordance with an embodiment of the invention.

FIG. 13 is a side perspective view of a foot depicting bone plates across a joint between first and second bones in accordance with an embodiment of the invention.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Examples of constructions, materials, and dimensions are provided for selected elements, and all other elements employ that which is known to those of ordinary skill in the field of the invention. Those skilled in the art will recognize that many of the noted examples have a variety of suitable alternatives.

Embodiments of the present invention include a bone cutting guide. In an exemplary application, embodiments of the bone cutting guide can be useful during a surgical procedure, such as a bone alignment, osteotomy, fusion procedure, and/or other procedures where one or more bones are to be cut. Such a procedure can be performed, for example, on bones (e.g., adjacent bones separated by a joint or different portions of a single bone) in the foot or hand, where bones are relatively smaller compared to bones in other parts of the human anatomy. In one example, a procedure utilizing the bone cutting guide can be performed to correct an alignment between a metatarsal (e.g. a first metatarsal) and a cuneiform (e.g., a first cuneiform), such as a bunion correction. An example of such a procedure is a Lapidus procedure. In another example, the procedure can be performed by modifying an alignment of a metatarsal (e.g. a first metatarsal). An example of such a procedure is a basilar metatarsal osteotomy procedure.

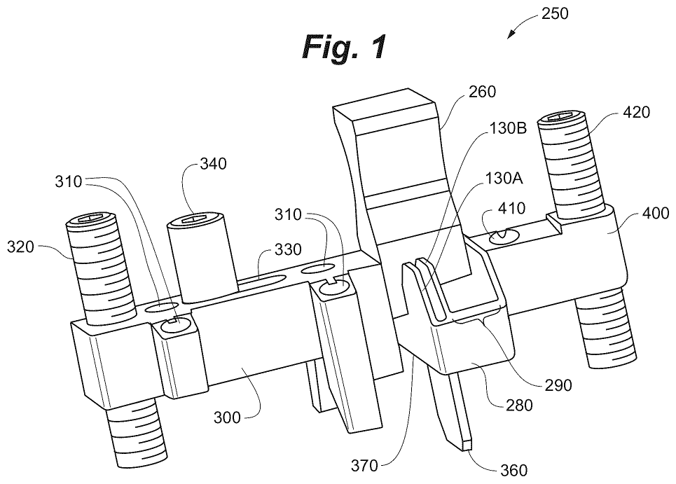

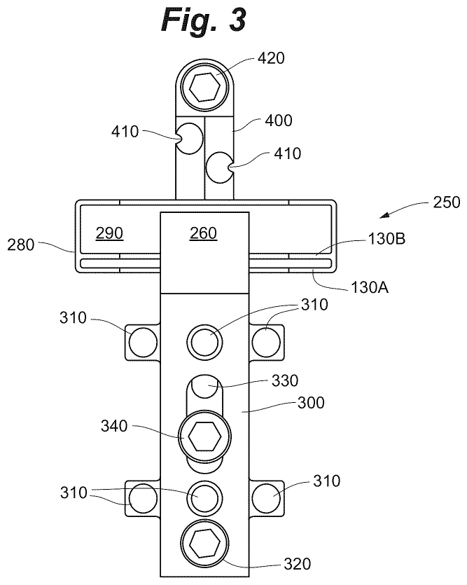

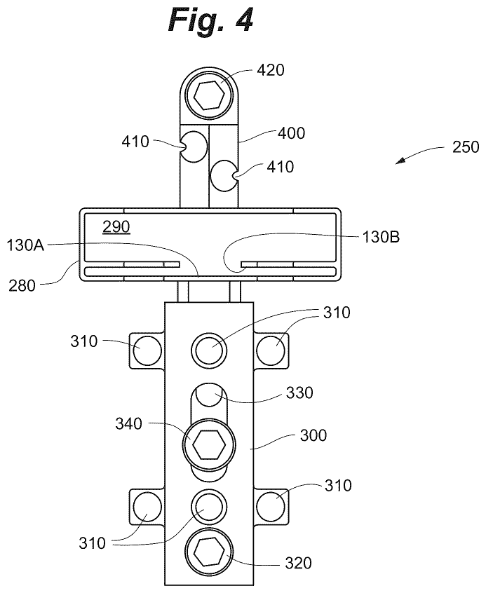

FIGS. 1-4 show an embodiment of a bone cutting guide 250. The bone cutting guide 250 includes a support 300 that defines an inner cavity. In one embodiment, the support 300 includes at least one fixation aperture 310 to receive at least one fixation pin. As shown, fixation apertures 310 can extend through the support 300 at a vertical angle (e.g., parallel to the longitudinal axis of the support) or a skewed angle relative to the longitudinal axis of the support (e.g., an angle ranging from 10 degrees to 55 degrees relative to the longitudinal axis of the support, such as an angle of approximately 20 degrees). In some configurations, the bone cutting guide 250 also includes an adjustable stabilization screw 320 engaged with the support 300 that can be used to stabilize the support with respect to a bone.

In the configuration of FIGS. 1-4, the bone cutting guide 250 includes a slot 330 and a securing component 340. The slot 330 is formed on and/or through at least a portion of a surface of the support 300. The securing component 340 is positioned at least partially within the slot 330 and configured to translate along the slot relative to the support 300. For example, the securing component 340 can have a first end with a diameter greater than a diameter of a second opposite end, such that the first end of the securing component 340 is supported by the slot 330 (e.g., the first end has a diameter greater than a width of the slot) while the second end of the securing component 340 is positioned within the slot (e.g., the second end has a diameter less than a width of the slot).

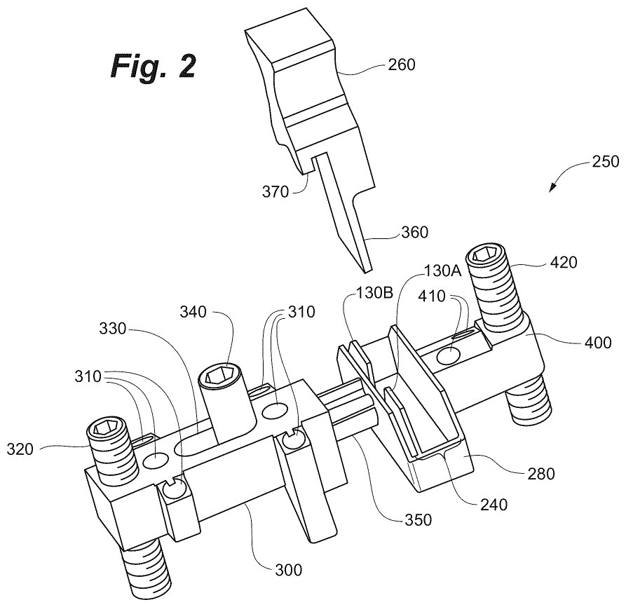

As shown best in FIG. 2, a shaft 350 can be positioned at least partially within the inner cavity of the support 300. The shaft 350 can be configured to translate within the inner cavity relative to the support 300, such that the shaft can project out from the inner cavity and retract into the inner cavity (compare shaft position in FIG. 1 to FIG. 2). In one embodiment, the securing component 340 can be threadingly engaged with the support 300 to bear against the shaft 350 to prevent the shaft 350 from traveling with the cavity when desired.

The bone cutting guide 250 in the illustrate example includes a main guide member 280 disposed on the shaft 350. In some embodiments, the main guide member 280 can be integral with the shaft, while in other embodiments the main guide member and the shaft can be separate components coupled together. The main guide member 280 can have a first guide surface 130A and, optionally, a second guide surface 130B. The first and second guide surfaces 130A and 130B can be adjacent surfaces facing one another with a space defined between the first and second guide surfaces 130A and 130B. In use, a clinician can position a cutting member (e.g., a saw) against first guide surface 130A (e.g., between first and second guide surfaces 130A and 130B) and translate the cutting member along or through the guide surface(s). In this way, the guide surface(s) can align the cutting member with the surface of a bone to be cut.

In the illustrated embodiment, the second guide surface 130B contains a gap bisecting the planar face of the second guide surface, such that the second guide surface 130B is not a single, continuous surface. This gap can be used by the clinician to visualize the cutting member when positioned between the first and second guide surfaces 130A and 130B. In other embodiments, the second guide surface 130B can be a single, continuous surface lacking any such gap.

As shown in FIGS. 1 and 2, the first guide surface 130A may define a first plane while the second guide surface 130B may define a second plane. The first guide surface 130A and the second guide surface 130B can be arranged such that the first plane is parallel to the second plane, with the space therebetween, as shown in FIGS. 1 and 2. Alternatively, the guide surfaces can be arranged such that the first and/or second planes are skewed (e.g., non-parallel relative to each other). Additionally, in some embodiments, the main guide member 280 includes a viewing window 290 to provide a visual path to bones during cuts.

In the embodiment shown in FIGS. 1 and 2, the cutting guide 250 includes a removable spacer 260 engageable with the main guide member 280. The spacer 260 can have a first portion 360 configured to extend into a joint space (e.g., in a joint space between a first metatarsal and medial cuneiform) and a second portion 370 engageable with the main guide member 280. Such a spacer can be useful for positioning the main cut guide at a desired position with respect to a joint. Further, the first and second surfaces of the main cut guide and/or the surfaces of the spacer can be used to establish a pre-determined cut thickness.

Some embodiments of the cutting guide 250 include an anchor 400 to connect to a bone portion that is spaced from a bone portion to which the support 300 is connected. For example, cutting guide 250 may bridge a joint, fracture, or cut with the adjustable stabilization screw 320 positioned on one bone portion (e.g., a metatarsal or cuneiform) and the anchor positioned on the opposite side (e.g., the other of the metatarsal or cuneiform). In some embodiments, the anchor 400 is translatable with the shaft 350 and located along the shaft 350 on a side of the main guide member 280 opposite the support 300.

In one embodiment, the anchor 400 includes at least one fixation aperture 410 to receive at least one fixation pin. Such aperture(s) may extend through the anchor at a parallel (e.g., vertical) or skewed angle relative to the longitudinal axis of the anchor. Further, as shown, a second adjustable stabilization screw 420 can be provided to stabilize the anchor with respect to a bone. In some embodiments, after making an angular correction to a bone, the anchor 400 can be used to hold the angular orientation of the bone so that a second cut can be made parallel to the first cut.

In practice, the bone cutting guide 250 can be used to guide one or more cutting operations performed on a bone or bones. For example, the bone cutting guide 250 can be used to cut the end faces of adjacent bones to prepare the end faces (e.g., leading edges) of the bones. Such adjacent end faces can end faces of two different bones separated by a joint, or can be different portions of a single bone, separated by a fracture. In some embodiments, a clinician may attach the bone cutting guide 250 to the bone or bones to be cut then advance a cutting member along one or more guide surfaces of the bone cutting guide to cut the end faces of the one or more bones. The clinician may realign the bones relative to each other before or after the bones are cut and may also perform additional surgical steps, such as bone plating, after the cuts have been made.

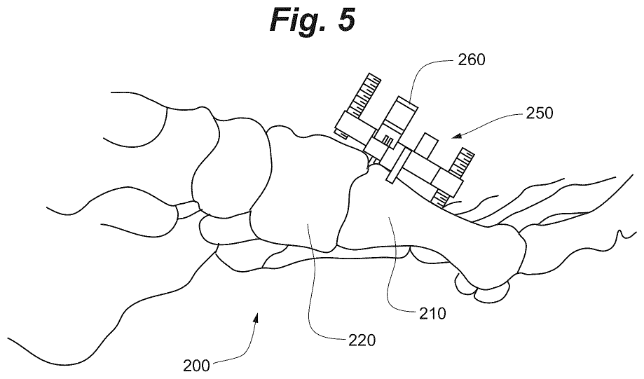

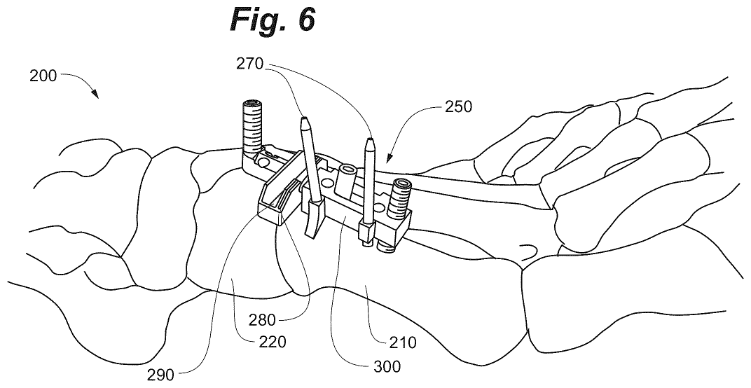

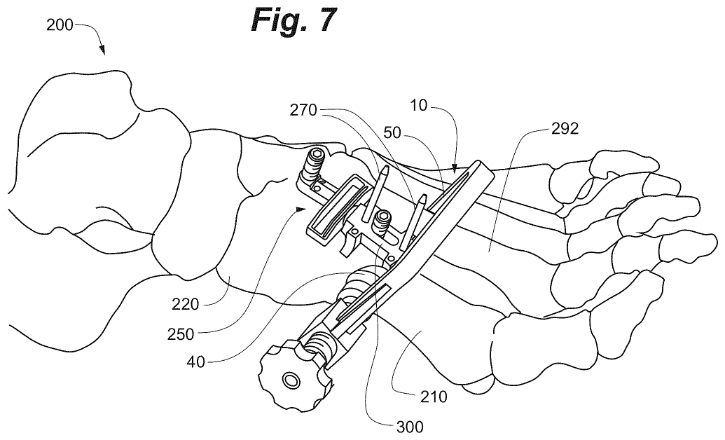

FIGS. 5-13 illustrate steps of an exemplary method for cutting a bone using a bone cutting guide, such as bone cutting guide 250 described with respect to FIGS. 1-4, during a medical procedure. FIGS. 5-13 depict a foot 200 having a first metatarsal 210 and a first cuneiform 220 (medial cuneiform). In FIG. 5, the bone cutting guide 250 is positioned in longitudinal alignment with the long axis of the first metatarsal 210 and the first cuneiform 220, generally on the dorsal or dorsal-medial surface. As shown, the spacer 260 can be positioned within the joint between the first metatarsal and the first cuneiform. As shown in FIG. 6, fixation pins 270 can be inserted into the support 300 of the bone cutting guide 250 through angled apertures to fix the position of the cutting guide 250 to the first metatarsal 210 and the spacer can be removed. The end of the first metatarsal 210 facing the first cuneiform 220 can be cut with a cutting member (e.g., saw) inserted through the main guide member 280 having parallel first and second surfaces. The main guide member can also include a viewing window 290 adjacent the first and second surfaces to facilitate visualization of the cutting procedure by the clinician. The cutting guide 250 can be removed vertically from the fixation pins 270 and the bone slice removed. As shown in FIG. 7, the cutting guide 250 can be inserted back on the foot 200, such as by inserting the fixation pins 270 through vertical apertures in support 300.

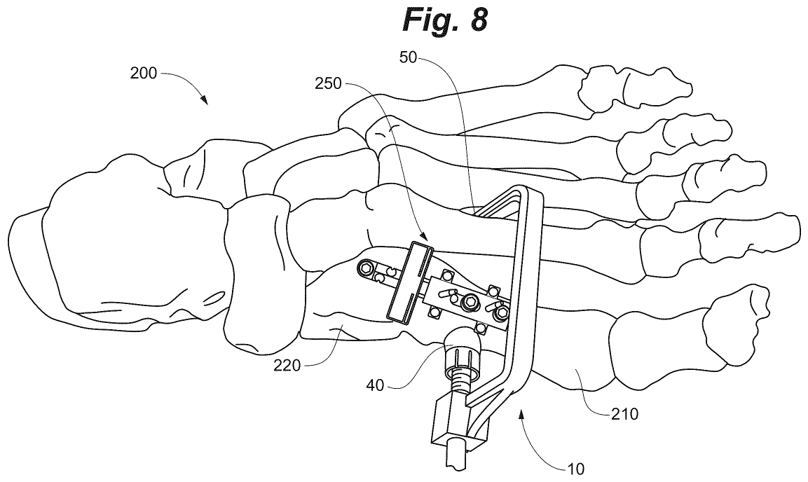

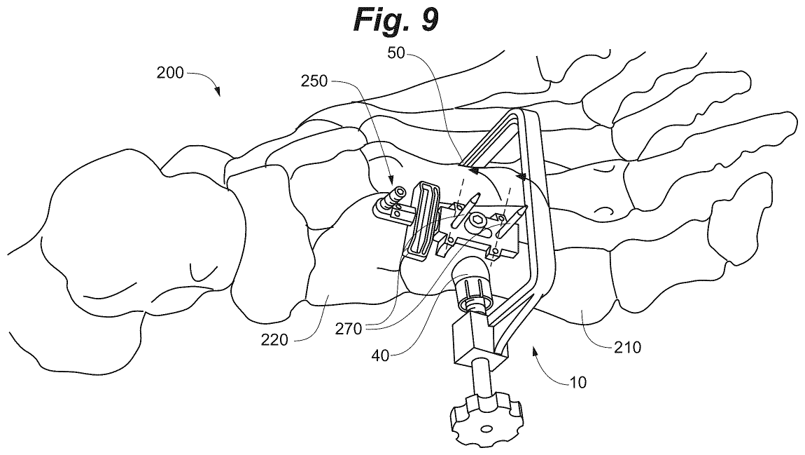

Also shown in FIG. 7, a bone positioning guide 10 can be attached to the first metatarsal 210 and adjacent second metatarsal 292 (e.g., by installing the bone positioning guide over the top of the cutting guide 250). In some examples, a concave surface of a bone engagement member 40 can be placed in apposition to a medial surface of the first metatarsal 210 and a tip 50 can be placed in apposition to a lateral side of a different metatarsal, such as a second metatarsal 292. FIG. 8 shows repositioning of the first metatarsal 210 with respect to the second metatarsal 292 by moving the bone engagement member 40 with respect to the tip 50 to correct a transverse plane deformity. FIG. 9 shows rotation of the first metatarsal 210 with respect to the bone engagement member 40 to correct a frontal plane deformity. In some embodiments, the fixation pins 270 can be used to impart rotational force to the first metatarsal 210, e.g., by the clinician grasping one or more of the pins and using the pins to physically manipulate the position of the first metatarsal 210. The bone positioning guide 10 can hold the desired position of the first metatarsal.

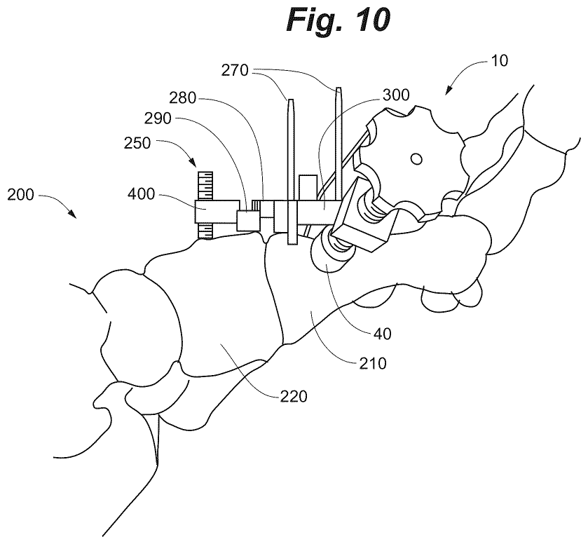

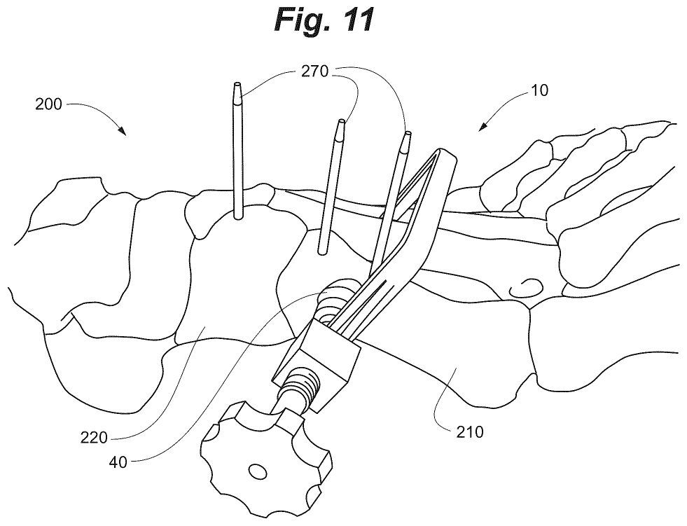

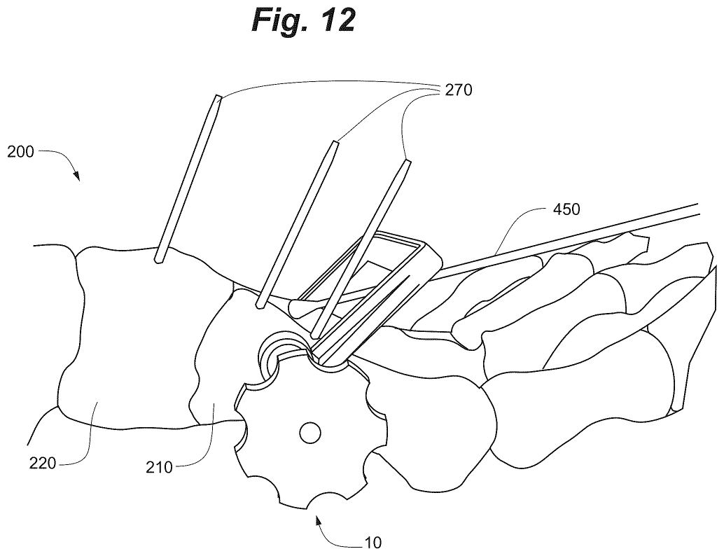

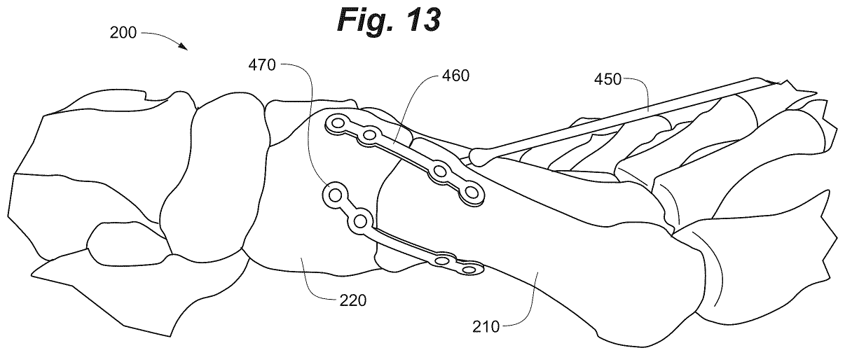

As shown in FIG. 10, the main guide member 280 can be extended from the support 300 to make a parallel cut in the first cuneiform 220. Additional fixation pins 270 can be inserted through apertures in the anchor 400 to fix the cutting guide 250 to the first cuneiform 220. A section of the cuneiform can be cut with a cutting member inserted through the main guide member 280 and removed. In some embodiments the cuneiform cut and the metatarsal cut are parallel, conforming cuts. As shown in FIG. 11, the cutting guide 250 can then be removed with the bone positioning guide 10 in place. FIG. 12 depicts a threaded olive pin 450 inserted through the first metatarsal 210 and into the first cuneiform 220 to provide compression between the first metatarsal and the first cuneiform. The bone positioning guide may then be removed. The position of the bones can then be fixed with a bone screw and/or one or more bone plates of any shape. FIG. 13 shows a first bone plate 460 (e.g., a straight or curved bone plate positioned on a dorsal-medial side) and a second bone plate 470 (e.g., a helical bone plate positioned from a medial side of the cuneiform to a plantar side of the metatarsal (other embodiments, not shown, may include a second straight or curved bone plate)) across the joint space. After the screws are inserted and/or the plates are applied with the insertion of bone screws, the fixation and olive pins may be removed.

Additional details on example surgical technical techniques that can be performed using a bone cutting guide in accordance with the disclosure, as well as details on example features that can be used in conjunction with such bone cutting guide, are described in U.S. patent application Ser. No. 14/981,335, entitled “BONE POSITIONING AND PREPARING GUIDE SYSTEMS AND METHODS” and filed on Dec. 28, 2015, and U.S. patent application Ser. No. 14/990,368, entitled “BONE PLATING SYSTEM AND METHOD” and filed on Jan. 7, 2016, the entire contents of both of which are incorporated herein by reference.

Thus, embodiments of the invention are disclosed. Although the present invention has been described with reference to certain disclosed embodiments, the disclosed embodiments are presented for purposes of illustration, and not limitation, and other embodiments of the invention are possible. One skilled in the art will appreciate that various changes, adaptations, and modifications may be made without departing from the spirit of the invention.

1. A bone cutting guide comprising:
a support defining an inner cavity;
a shaft disposed at least partially within the inner cavity, wherein the shaft is translatable within the inner cavity relative to the support; and
a main guide member located along the shaft, wherein the main guide member includes a first guide surface defining a first plane.

2. The bone cutting guide of claim 1, further comprising a spacer engageable with the main guide member.

3. The bone cutting guide of claim 1, wherein the main guide member further comprises a viewing window.

4. The bone cutting guide of claim 1, further comprising an anchor translatable with the shaft on a side of the main guide member opposite the support.

5. The bone cutting guide of claim 4, further comprising a first adjustable stabilization screw engaged with the support and a second adjustable stabilization screw engaged with the anchor.

6. The bone cutting guide of claim 4, further comprising at least one fixation aperture extending through the support, wherein at least one fixation pin is configured to extend through the first fixation aperture to fixate the support, and at least one fixation aperture extending through the anchor, wherein at least one fixation pin is configured to extend through the second fixation aperture to fixate the anchor.

7. The bone cutting guide of claim 1, wherein the main guide member includes a second guide surface defining a second plane.

8. The bone cutting guide of claim 1, wherein the second plane is parallel to the first plane.

9. A bone cutting guide comprising:
a support defining an inner cavity;
a shaft disposed at least partially within the inner cavity, wherein the shaft is translatable within the inner cavity relative to the support;
a main guide member located along the shaft, wherein the main guide member includes a first guide surface defining a first plane, the main guide member comprising a viewing window;
an anchor translatable with the shaft on a side of the main guide member opposite the support; and
a first adjustable stabilization screw engaged with the support and a second adjustable stabilization screw engaged with the anchor.

10. A method for cutting one or more bones, the method comprising:
fixing a support to a bone;
aligning a main guide member to be positioned at a location to be cut; and
making a first cut at the location to be cut by aligning a cutting member with a first guide surface of the main guide member.

11. The method of claim 10, wherein aligning the main guide member to be positioned at the location to be cut comprises the step of:
inserting a spacer into a joint and adjusting a location of the main guide member relative to a longitudinal axis of the bone.

12. The method of claim 10, further comprising the step of:
aligning the main guide member to be positioned at a second location to be cut; and
making a second cut at the second location to be cut by aligning the cutting member with the first guide surface of the main guide member.

13. The method of claim 10, further comprising the step of:
correcting an alignment of the bone.
